(19) United States
(12) Patent Application Publication
(10) Pub. No.: US 2019/0036992 A1
(43) Pub. Date: Jan. 31, 2019

(54) SURGICAL COMMUNICATIONS SYSTEMS AND RELATED METHODS

(71) Applicants: Charles D. Stewart, Provo, UT (US); Adam Helland, Tucson, AZ (US)

(72) Inventors: Charles D. Stewart, Provo, UT (US); Adam Helland, Tucson, AZ (US)

(21) Appl. No.: 16/035,163

(22) Filed: Jul. 13, 2018

Related U.S. Application Data
(60) Provisional application No. 62/517,710, filed on Jun. 9, 2017.

(51) Int. Cl. H04L 29/06; H04B 1/3827; H04N 5/262; H04N 7/18; A61B 90/35; A61B 90/00

(52) U.S. Cl. CPC H04L 65/4069; H04B 1/385; H04N 5/2628; H04L 67/02; A61B 90/35; A61B 90/361; H04N 7/183

(57) ABSTRACT

Methods and systems for communication during a procedure (e.g., a medical or surgical procedure) are provided. In one embodiment, a wearable headset is provided that includes a camera. The headset may additional include a headlamp. The camera may be configured for wireless transmission of video to a display device for viewing by an assistant present during the procedure or to a viewer that is remotely located. In one particular example, the video may be transmitted using HTML protocols in real time, such that the lag time is largely unrealized by a person viewing the transmitted data on a display device. In one embodiment, the transmitted image is “flipped” or reversed left-to-right for the benefit of a person assisting in the procedure.

### RELATED APPLICATION

This application claims the benefit of the filing date of U.S. Provisional Application No. 62/517,710 and entitled SURGICAL COMMUNICATIONS AND RELATED METHODS, pending, the disclosure of which is incorporated in its entirety by this reference.

### TECHNICAL FIELD

The present disclosure related generally to systems and methods of real time communications systems, including video, which may be used, for example, in conjunction with surgical or other medical procedures.

### BACKGROUND

In a surgical settings, communication is a necessary component among the team members, whether the team members are present in the same location (e.g., the same surgical room), or whether they are dispersed at various locations. The communication between team members, and the communication of actual situations and conditions to individual team members, is imperative for the team members to work together cohesively, fluently and efficiently.

One example demonstrating the need for accurate and efficient communication on various levels is that of performing the plastic surgery procedure known as a face lift (technically known as a rhytidectomy). A face lift procedure conventionally involves the removal of excess facial skin (e.g., skin forming as wrinkles). During the procedure various incisions are made (e.g., in front of the ear extending up into the hairline). After the skin incision is made, the skin may be undermined by separating it from deeper tissues with a scalpel or scissors. After the skin is separated from the deeper tissues, the deeper tissues can be tightened with sutures. Alternatively, or additionally, some of the excess deeper tissue may be removed. The skin is then redraped with the amount of excess skin to be removed being determined by the surgeon. After removal of the excess skin, the remaining skin incisions are closed with sutures and/or staples.

During the process, due to physical limitations, only the surgeon is conventionally able see beneath the skin while any work is being done (e.g., during undermining, deep tissue tightening, etc.). An assistant may pull the skin tight and away from the facial structure during the procedure to aid the surgeon during the process. However, the assistant is not typically able to see the surgeon's actions beneath the skin since the skin acts as a visual barrier to the surgeon's actions relative to the assistant. Thus, the surgeon has to provide verbal commands to the assistant regarding any actions that need to be taken since he/she is the only one able to assess the actions being taken or the conditions that prevail beneath the skin during the procedure.

In another example regarding the desirability of enhanced communication during a surgical or medical procedure, it is often desirable to provide real time information regarding the progress of a procedure, or the actual conditions present during the procedure, to someone that is offsite. The offsite team member, having up-to-date information regarding the procedure may then provide valuable and timely insight and direction during the procedure.

In yet another example, it may also be desirable to provide real time information regarding a procedure to a group of individuals for purposes of teaching or training.

It is a desire within the industry to provide systems and methods and enhance communication among multiple parties during a surgical or medical procedure to provide enhanced quality of medical treatment.

### BRIEF DESCRIPTION OF THE DRAWINGS

The foregoing and other advantages of the invention will become apparent upon reading the following detailed description and upon reference to the drawings in which:

FIG. 1 is a schematic diagram showing a communications system according to an embodiment of the present disclosure;

FIG. 2 is a perspective view of a headset in accordance with an embodiment of the present disclosure;

FIG. 3 is a block diagram of a system including the headset shown in FIG. 2;

FIGS. 4A and 4B are front and rear views of a head-up display device that may be used in accordance with certain embodiments of the present disclosure; and

FIG. 5 diagram showing a wireless device according to an embodiment of the present disclosure.

### DETAILED DESCRIPTION

Embodiments of the present disclosure include communications systems, methods and apparatuses that may be used in conjunction with time-critical activities such as, for example, surgical and medical procedures.

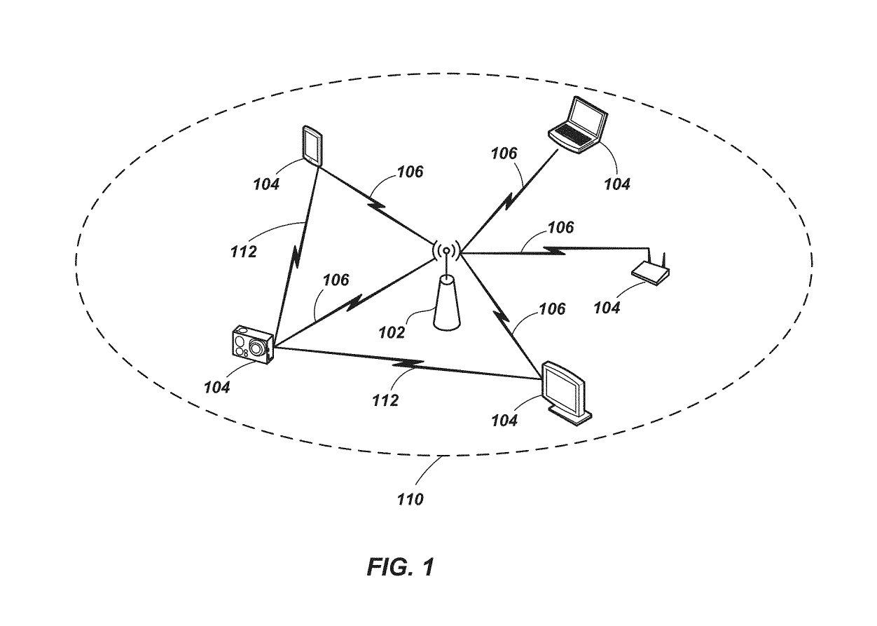

Referring to FIG. 1, a diagram illustrates an example of a wireless local area network (WLAN) 100 that may be employed in accordance with an embodiment of the invention. The WLAN network 100 may include an access point (AP) 102 and one or more wireless devices or stations (STAs) 104, such as mobile stations, personal digital assistants (PDAs), other handheld devices, netbooks, notebook computers, tablet computers, laptops, display devices (e.g., TVs, computer monitors, etc.), printers, digital cameras (video or still cameras), etc. While only one AP 102 is illustrated, the WLAN network 100 may have multiple APs 102. Each of the wireless stations 104, which may also be referred to as mobile stations (MSs), mobile devices, access terminals (ATs), user equipment (UE), subscriber stations (SSs), or subscriber units, may associate and communicate with an AP 102 via a wireless communication link 106. Each AP 102 has a geographic coverage area 110 such that wireless stations 104 within that area can typically communicate with the AP 102. The wireless stations 104 may be dispersed throughout the geographic coverage area 110. Each wireless station 104 may be stationary or mobile.

Although not shown in FIG. 1, a wireless station 104 can be covered by more than one AP 102 and can therefore associate with one or more APs 102 at different times. A single AP 102 and an associated set of wireless stations 104 may be referred to as a basic service set (BSS). An extended service set (ESS) is a set of connected BSSs. A distribution system (DS) (not shown) is used to connect APs 102 in an extended service set. A geographic coverage area 110 for an access point 102 may be divided into sectors making up only a portion of the coverage area (not shown). The WLAN network 100 may include access points 102 of different types (e.g., metropolitan area, home network, etc.), with varying sizes of coverage areas and overlapping coverage areas for different technologies. Although not shown, other wireless devices can communicate with the AP 102.

While the wireless stations 104 may communicate with each other through the AP 102 using communication links 106, each wireless station 104 may also communicate directly with one or more other wireless stations 104 via a direct wireless link 112. Two or more wireless stations 104 may communicate via a direct wireless link 112 when both wireless stations 104 are in the AP geographic coverage area 110 or when one or neither wireless station 104 is within the AP geographic coverage area 110. Examples of direct wireless links 112 may include Wi-Fi Direct connections (also known as peer-to-peer (P2P) connections), connections established by using a Wi-Fi Tunneled Direct Link Setup (TDLS) link, and other P2P group connections. The wireless stations 104 in these examples may communicate according to the WLAN radio and baseband protocol including physical and media access control (MAC) layers. In other implementations, other peer-to-peer connections and/or ad hoc networks may be implemented within WLAN network 100.

In some examples, one or more of wireless stations 104 may be configured as a source device and/or a sink device. For example, a source device (e.g., a first wireless station 104) may be connected to a sink device (e.g., a second wireless station 104) via a unidirectional communication channel or link that may be a wireless link in some embodiments. Communications between a source device and a sink device, connected via a wireless peer-to-peer connection, may be configured to remotely render content of the source device at the sink device(s). In some examples, the unidirectional communication link between the source device and the sink device may allow users to launch applications stored on the source device via the sink device. For example, the sink devices may include various input controls (e.g., mouse, keyboard, knobs, keys, user interface buttons). These controls may be used at the sink device to initialize and interact during the audio/video streaming from the source through the media applications stored on the source device.

In some examples, the source device may be connected to the sink device via a Wi-Fi Display connection. Wi-Fi Display protocol, which may be known as Miracast® by Wi-Fi alliance, allows a portable device or computer to transmit media content (e.g., video, audio, images, etc.) to a compatible display wirelessly. It enables delivery of compressed standard, high-definition, or ultra-high definition video content along with audio in various formats over a unidirectional communication link. It also may allow users to echo the display from one device onto the display of another device. The unidirectional communication link may be a direct wireless link (e.g., peer-to-peer link), or an indirect wireless link through a Wi-Fi access point 102. Examples of direct wireless links include Wi-Fi Direct connections and connections established by using a Wi-Fi Tunneled Direct Link Setup (TDLS) link. Additionally, wireless remote display may also include, but is not limited to the Wi-Fi Display specification, also known as, Discovery and Launch (DIAL), Digital Living Network Alliance® (DLNA), Airplay, WirelessHD, Wireless Home Digital Interface (WHDI), Intel's Wireless Display (Wi-Di) technology, MirrorLink technology, and Ultra-wideband (UWB) connections.

In one example of communications between a sink device and a source device, a sink device (e.g., a first wireless station 104 configured to act as a sink device) may identify a unidirectional communication channel with a source device. The sink device may determine that a trigger associated with particular type of transmission to the source device has been activated (e.g., activated by the sink device or the source device). In some examples, the trigger may be associated with one or more capabilities or parameter support messages exchanged between the sink device and the source device. The sink device may initiate the transmission to the source device based on the trigger being identified or detected. For example, the sink device may send one or more packets containing audio or video information to the source device.

A source device (e.g., a second wireless station 104 configured to act as a source device) may receive an indication that the sink device supports a particular type of transmission via the unidirectional communication channel, an indication of various parameters for the specified type of transmission supported by the sink device, and the like. The sink device may send a trigger to the source device to initiate the transmission via the unidirectional communication channel and then receive the transmission from the sink device based on the trigger. Such a process may be used, for example, to transmit audio, video, data, control signaling, etc., between such devices.

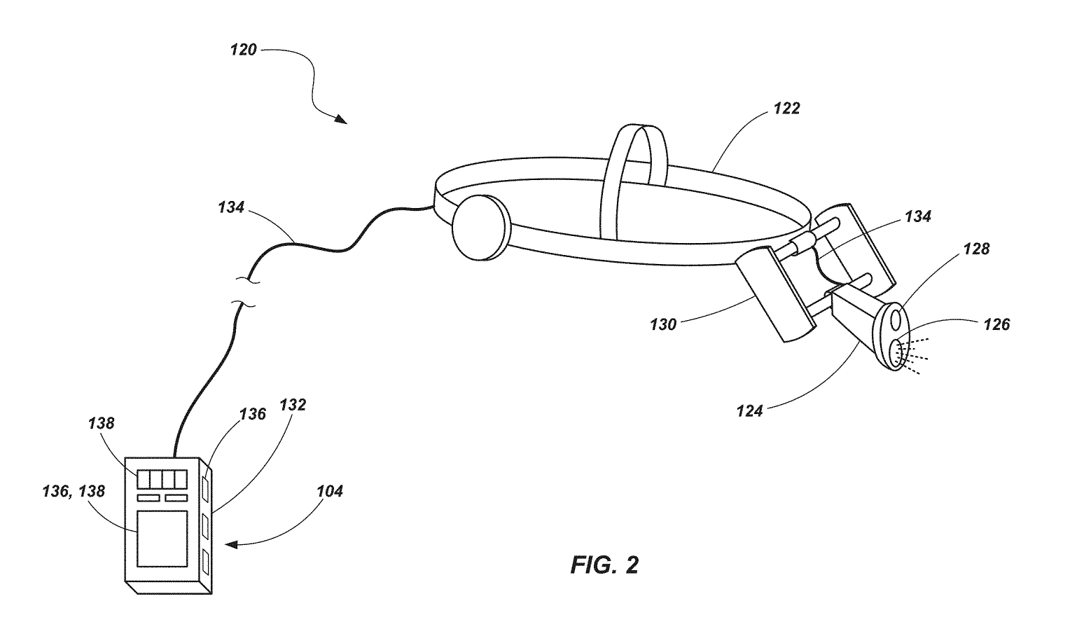

Referring to FIG. 2, a headset 120 is shown in accordance with an embodiment of the present disclosure. The headset 120 may include, or be associated with, a wireless station 104 such as described in association with FIG. 1. The headset 120 may include an adjustable headband 122 configured to fit the head of a surgeon or other member of a medical team. Attached to the front side of the headband is a housing 124 having a lamp 126 (e.g., an LED lamp or other appropriate light) and a video camera 128. While the embodiment shown in FIG. 2 depicts the lamp 126 and camera 128 being contained in a common housing 124, the lamp 126 and camera 128 may exist in separate housings and be separately mounted to the headband 122. The lamp 126 provides illumination on a subject or target area for both the surgeon or other practitioner as well as for the video camera 128. The housing 124 may be coupled to the headband 122 by means of an adjustable mechanism 130 in order to direct the lamp 126 and the camera 128 with respect to the subject that is being illuminated by the lamp 126 and the image being captured by the camera 128. In other embodiments, the lamp 126 and the camera 128 may be individually adjustable relative to the housing 124, or individually adjustable relative to the headband 112 when provided as separate devices.

The lamp 126 and camera 128 may be coupled with a control unit 132, such as by way of an electrical cable 134, to turn the lamp 126 and/or camera 128 on and off, or to control such components in other ways (e.g., control the intensity of the lamp 126, initiate or end image capture by the camera 128, etc.). Thus, the control unit 132 may include one or more input devices 136 such as switches, buttons, touchscreens, dials, etc. to effect such control of the lamp 126, the camera 128 or both.

A battery pack (not shown) may be associated with the control unit 132 to provide power to the lamp 126 and camera 128. The battery pack may be replaceable and/or rechargeable. Of course, other sources of power are also contemplated as being used. In one embodiment, the control unit 132 may further include a wireless station 104 to transmit information from the camera 128 to another device. The wireless station 104 associated with the headset 120 may transmit live video to one or more other wireless station(s) for viewing by individuals who are either located near the individual wearing the headset, but have an obstructed view of the image being captured by the camera, or who are remotely located (e.g., in another room, building, or even thousands of miles away). In other embodiments, the wireless station 104 may be located somewhere other than with the control unit 132 (e.g., with the camera 128).

The control unit 132 may further include output devices 138 such as displays, or other indicators to provide feedback to a user. For example, indicators may show that a particular device (e.g., the lamp 126 or the camera 128) is turned on and functioning, what the status of the power source is, or if the associated wireless station 104 is transmitting data or in communication with an AP 102 or other wireless station 104. Such indicators might include, for example, lights, LCD or LED displays, and/or audio signals.

The wireless station 104 associated with the headset 120 may be configured to stream live video to a display devices for viewing by an assistant or a remote individual using, for example, a WLAN such as described above, or using other appropriate methods. In one embodiment, the wireless station 104 associated with the headset 120 transmits video and/or other data to another wireless station 104 associated with a display device 132 via the WLAN 100 in real time, meaning that there is little, if any perceivable lag time in the transmission. For example, in one embodiment, a video stream may be transmitted from the camera to a display device at a frame rate of 30 frames per second while having approximately 200 milliseconds (ms) or less of latency. This provides a video stream with effectively imperceptible latency to a user and without video stuttering.

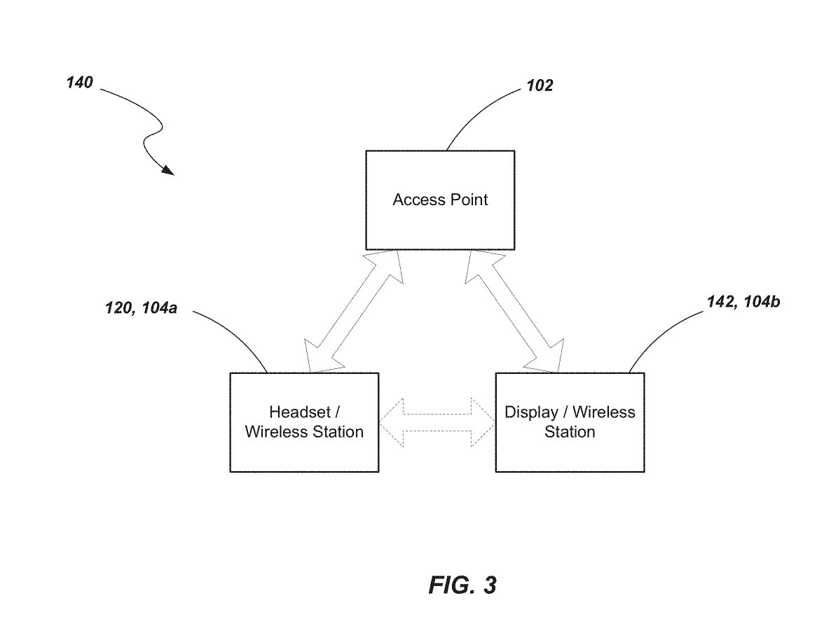

Referring to FIG. 3, a block diagram of a communications system 140 is shown using a headset 120 or other like communications device. The system 140 shown in FIG. 3 includes the headset 120 having a camera 128 and a wireless station 104a. As noted above, the wireless station 104a may be configured to transmit a wireless signal that is associated with video data (and, optionally, other types of data). The wireless station 102 may transmit data by way of an AP 102 or other communications device to one or more display devices 142 associated with another wireless station 104b. In other embodiments, the first wireless station 104a may communicate directly with the second wireless station such as has been described above.

The display(s) 142 may include any of a variety of devices. For example, in one embodiment, the display 142 may include a dedicated video display (e.g., a VGA display, an LCD or LED monitor or similar device). In another embodiment, the display 142 may be display associated with a device that is configured to perform various computing tasks (e.g., a laptop computer, a tablet-form computing device such as an iPad® or some other mobile computing device). In yet another example, the display 142 may include a wearable device, sometimes referred to as a personal video device, such as Google Glass or a Microsoft Hololens device. In yet another embodiment, a personal video device (e.g., Google Glass) may be used to function as the camera/headset. In one embodiment, both the camera/headset and the display device may include personal video devices. Examples of Google Glass and Hololens type devices are described in U.S. Pat. No. 8,203,502 to Chi et al., U.S. Pat. No. 8,874,988 to Geisner et al., and U.S. Patent Application Publication No. 20130044042 to Olsson et al., the disclosures of which are incorporated by reference herein in their entireties.

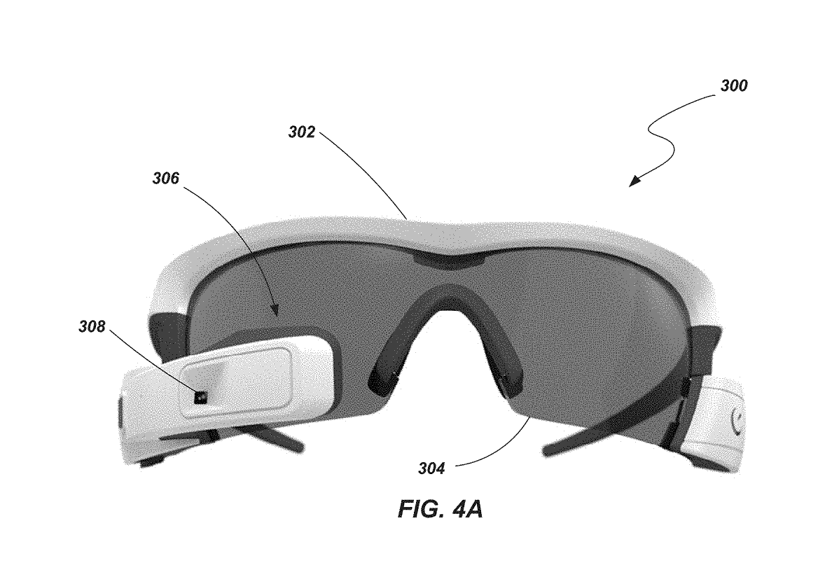

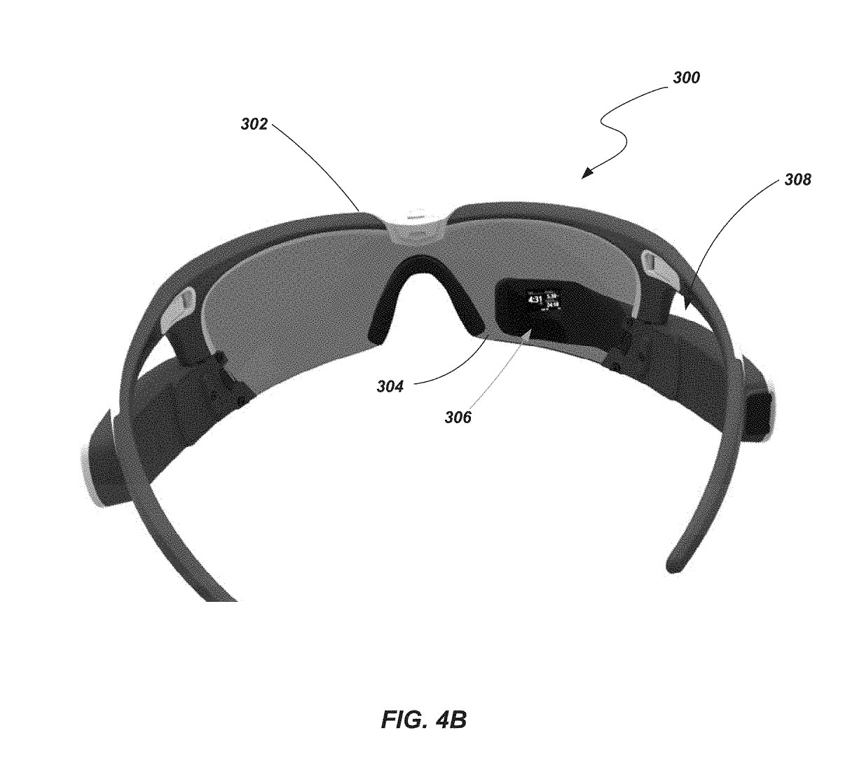

Other examples of a personal display device include “head-up” type displays, sometimes referred to as “in-sight” displays. Some specific examples of a head-up display include those offered by Recon Instruments of Vancouver, BC, including the products currently being offered as the Recon Jet™ and the Recon Jet Pro™. In the example of the Recon Instruments type products, a pair of glasses includes a display positioned just below the wearer's right eye. For example, referring to FIGS. 4A and 4B, a head-up display device 300 (the Recon Jet model being shown) may include a frame 302, lenses 304 (which may be clear or tinted depending on certain applications), and a display 306. In one example, the display 306 “projects” a virtual image that appears as if it were a 30 inch high definition display being viewed from 7 feet by the viewer/wearer. Additionally, similar to embodiments described above, the head-up display device 300 may include a camera 308. Another example of a head-up or in-sight personal display device includes the Varia Vision™ device commercially available from Garmin International, Inc. of Olathe, Kans.

In one accordance with one embodiment of the invention, the image that is transferred to a display 142 may be horizontally “flipped” or reversed such that, for example, what a surgeon sees on the right hand side (and the image that is captured by the camera 128 as being on the right hand side) of a given scene is displayed as being on the left hand side of the display 142, and vice versa.

During a surgical or medical procedure, the communications system 140 may be used, for example, by positioning the camera 128 to capture a video image of the scene that is being viewed by the individual wearing the headset 120. The image is transmitted, or streamed, to a display 142 for viewing in real time (e.g., with approximately 200 ms latency or less) by another party. In one particular example, the user wearing the headset 120 may be a plastic surgeon performing a face lift or other type of operation. In such a case, an assistant (e.g., a nurse or fellow surgeon) may have their view occluded with regard to certain actions of the surgeon or various anatomical features of the patient such as those that may lie below the skin of the patient's face. This limited or occluded view can be overcome by the assistant's viewing of the transmitted video on the display 142.

Additionally, in the example of a face lift, the assistant may be positioned so that they are directly across from and facing the surgeon while they provide their assistance. By horizontally “flipping” or reversing the image, the assistant may track the movements of the surgeon and assess or predict the needs of the surgeon while certain actions or procedures are being performed. The flipped or reversed image will further correlate with the assistant's “left/right” orientation of actions being taken by the surgeon during the procedure when the assistant and surgeon are facing in one another, such as when an assistant is holding or pulling pack the patient's skin during a face lift.

As previously noted, the video data may be transmitted to locations that are remote from the performance of the medical procedure, whether for real time consulting by another practitioner or for educational purposes. Such remote transmittal may occur through broader network such as the internet.

One particular example of a system 140 is described in Appendix A which describes a prototype example using a Raspberry Pi based computer to send video to a display via a wireless access point. The video was transmitted using an HTML format enabling transmission at up to 30 frames per second with less than 200 ms lag time.

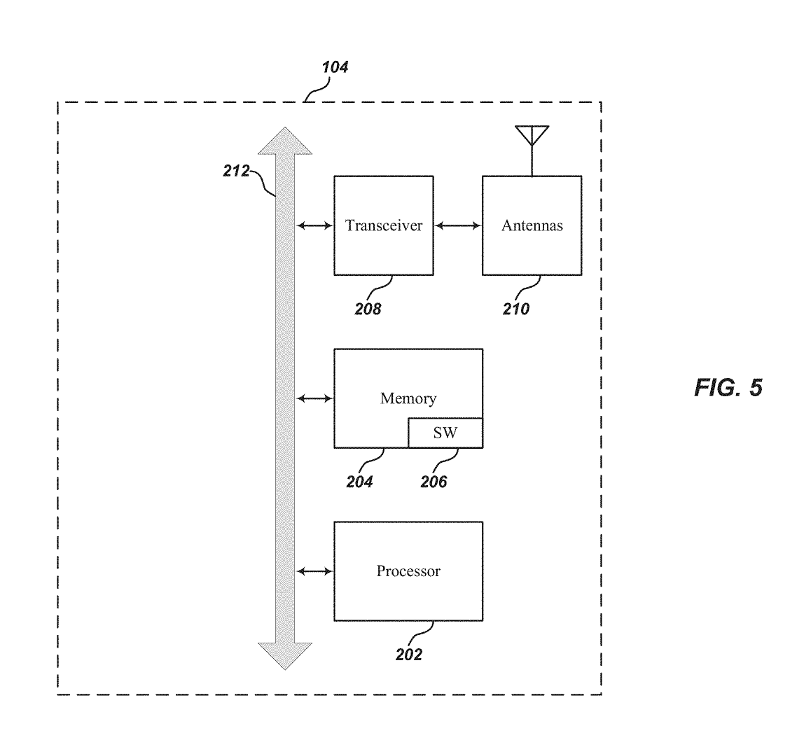

Referring now to FIG. 5, a block diagram is shown of an example of a wireless device 200 according to various aspects of the present disclosure. The wireless device 200, which may be an example of a wireless station 104 such as described above with reference to FIGS. 1-3. The wireless device 200 may include components for bi-directional communications including components for transmitting communications and components for receiving communications. The wireless device 200 may also include a processor 202, and memory 204 (including software (SW) 206), a transceiver 208, and one or more antenna(s) 210, each of which may communicate, directly or indirectly, with one another (e.g., via buses 212). The transceiver 208 may communicate bi-directionally, via the antenna(s) 210 or wired or wireless links, with one or more networks, as described above. For example, the transceiver 208 may communicate bi-directionally with access point 102 or another wireless station 104. The transceiver 208 may include a modem to modulate the packets and provide the modulated packets to the antenna(s) 210 for transmission, and to demodulate packets received from the antenna(s) 210. While the wireless device 200 may include a single antenna 210, wireless device 200 may also have multiple antennas 210 capable of concurrently transmitting or receiving multiple wireless transmissions.

The memory 204 may include random access memory (RAM) and read only memory (ROM). The memory 204 may store computer-readable, computer-executable software/firmware code 206 including instructions that, when executed, cause the processor 202 to perform various functions described. Alternatively, the software/firmware code 206 may not be directly executable by the processor 202 but cause a computer (e.g., when compiled and executed) to perform various functions. The processor 202 may include an intelligent hardware device, (e.g., a central processing unit (CPU), a microcontroller, an ASIC, etc.)

Further, as above, the wireless stations 104 may be connected to a broader network, such as the internet (e.g., the WLAN may be connected to the internet). Thus, the system may be used to facilitate communication of data (e.g., including video and or audio) locally, remotely, or both simultaneously.

It is noted that any feature or element of one described embodiment may be combined with any other embodiment without limitation. While the invention may be susceptible to various modifications and alternative forms, specific embodiments have been shown by way of example in the drawings and have been described in detail herein. However, it should be understood that the invention is not intended to be limited to the particular forms disclosed. For example, while systems may have been specifically described in terms of wireless communications, it is noted that various components may be in “wired” communication (e.g., a camera may be connected to a display using a wired VGA, DVI or HDMI connection). Thus, the invention includes all modifications, equivalents, and alternatives falling within the spirit and scope of the invention as defined by the following appended claims.

What is claimed is:

1. A system comprising:
a headset including a camera;
a first wireless device associated with the camera;
a display device;
a second wireless device associated with the display device;
wherein the camera and first wireless device are configured to wirelessly transmit a video stream to the display device via the second wireless device, and wherein the display device is configured to present the video stream as a reversed image.

2. The system of claim 1, wherein the display device comprises a wearable, personal display device.

3. The system of claim 1, wherein the headset further includes a lamp.

4. The system of claim 3, wherein the camera and the lamp are coupled with a battery pack.

5. The system of claim 1, wherein the camera and the first wireless device are configured to transmit the video stream using a hypertext mark-up language (HTML) format.

6. The system of claim 1, wherein the camera and the first wireless device are configured to transmit the video stream for display at approximately 30 frames per second.

7. The system of claim 1, wherein the lag time between capturing an image by the camera and displaying the image on the display device is approximately 200 milliseconds or less.

8. The system of claim 1, further comprising a wireless access point, wherein the first wireless device and the second wireless device communicate via the wireless access point.

9. A method of performing a medical procedure, the method comprising:
placing a headset on a head of a practitioner, the headset including a camera;
capturing video images of a target area of interest on the patient with the camera;
wirelessly streaming the captured video to a display device;
reversing the captured image for viewing by another party on the display device.

10. The method according to claim 9, further comprising displaying the reversed, captured image on the display device with a lag time of approximately 200 milliseconds or less.

11. The method according to claim 9, further comprising providing the headset with a lamp and illuminating the target area of interest with the lamp.

12. The method according to claim 9, wherein the target area of interest is at least partially blocked from view of the another party.

13. The method according to claim 9, wherein the practitioner includes a surgeon and the another party includes an assistant.

14. The method according to claim 9, wherein wirelessly streaming the captured video to a display device includes wirelessly streaming the captured video to a wearable, personal video display device.

15. The method according to claim 9, wherein the medical procedure includes performing a face lift.
